(19) United States
(12) Patent Application Publication
(10) Pub. No.: US 2007/0086963 A1
(43) Pub. Date: Apr. 19, 2007

(54) APPARATUS TO APPLY A HAIR CARE PREPARATION

(75) Inventors: Ruth Stern, Toronto (CA); Pauline Ashworth, Toronto (CA); Mark Harnden, Cobourg (CA)

Correspondence Address: BERESKIN AND PARR, 40 KING STREET WEST, BOX 401, TORONTO, ON, M5H 3Y2, CA

(73) Assignee: Colour Revolution Inc., Toronto (CA)

(21) Appl. No.: 11/538,855

(22) Filed: Oct. 5, 2006

Related U.S. Application Data
(63) Continuation-in-part of application No. 11/371,944, filed on Mar. 10, 2006.
Application No. 11/371,944 is a continuation-in-part of application No. 11/145,068, filed on Jun. 6, 2005.

(51) Int. Cl. A61K 8/00

(52) U.S. Cl. 424/62; 8/405

(57) ABSTRACT

A method for coloring hair with a hair care formulation that is mixed prior to use is provided. The method includes mixing together the components of the hair care formulation as the formulation is dispensed from a plastic applicator having at least one storage compartment for each component and a static or motionless mixer.

This application is a continuation-in-part of U.S. application Ser. No. 11/371,944 which was filed on Mar. 10, 2006 and is still pending, which is a continuation-in-part of U.S. application Ser. No. 11/145,068 which was filed on Jun. 6, 2005 and is still pending.

### FIELD OF THE INVENTION

This invention relates to a method of treating hair. In a preferred aspect, the application relates to a method of coloring the new growth of hair that has been previously coloured.

### BACKGROUND OF THE INVENTION

The most common method of dyeing human hair is oxidative dyeing. Various formulations for use in oxidative hair dyeing or coloring are well known in the art. Such formulations typically involve the use of a two-part system. One part, the dye component, contains at least one primary intermediate and at least one coupler. For example, the dye component may contain diaminobenzenes, dihydroxybenzenes and aminophenols. Before use, this dye component is mixed with a second part, which is a developer formulation containing an oxidizing agent, such as hydrogen peroxide or other strong oxidizing agent. The developer oxidizes the primary intermediate (e.g. the benzenes), which then reacts with the coupler (e.g. the aminophenols) to form a colored compound. See for example U.S. Pat. No. 6,565,615 (Wong et al).

Typically, the dye component and the developer are mixed shortly prior to use. If a person has their hair dyed in a hair salon, then the hair coloring formulation is typically mixed by a hairdresser just prior to the application of the hair coloring formulation. Home hair coloring formulations are sold to the general public. Typically, these must also be mixed prior to use and, accordingly, an individual consumer must mix these formulations prior to use. Such formulations may be used to dye all or part of the hair of the person.

A common problem with using long-lasting hair dyes is that as a person's hair grows, the new hair that emerges from the scalp is uncolored. Accordingly, in some cases, the person will mix a hair coloring formulation to color the new hair instead of re-coloring all of their hair.

Hair coloring formulations may irritate the scalp of a user. Accordingly, it is typically recommended that a part of the hair coloring formulation be applied to the skin of a user up to 48 hours prior to the use of the formulation to color the person's hair to test whether the hair coloring formulation will not irritate the scalp. Accordingly, at least a part of the formulation must be decanted from its container and mixed so that the formulation must be tested on the person's skin. Typically, hair-coloring formulations must be utilized within about 45 minutes of being mixed. Therefore, a portion of the formulation must be decanted and mixed while the rest is stored for subsequent decanting and mixing, provided that the skin of the person does not react too much to the formulation.

Various types of containers for storing two components of a mixture are known in the art. These include, for example, U.S. Pat. Nos. 2,819,723; 3,570,499; 5,725,499 and 6,036,005.

U.S. Pat. Nos. 5,725,499 (Silverstein et al) discloses a dual barreled syringe, which may be used for storing and dispensing two components of a formulation. The formulation may be mixed once dispensed from the syringe or as it is dispensed from the syringe dual barreled syringe. It is known that a dual barreled syringe may be used to dispense a hair coloring formulation.

U.S. Pat. Nos. 2,819,723 (Mayer) and 3,570499 (Ruta) disclose applicators for applying a hair treatment preparation. The applicators have two different compartments, which store different components of the hair treatment formulation. The different components are mixed as they are dispensed from the applicators. The applicators include comb or brush portions so that, once the hair treatment formulation is mixed, it may be brushed into the hair of a person.

U.S. Pat. No. 5,339,839 (Forcelledo et al) and 6,260,557 (Yarbrugh) disclose combs, which have passages extending from a base portion to the tip of the teeth of the comb, so that a hair coloring formulation may be passed through the comb and dispensed at the teeth of the comb.

A disadvantage with these devices is that the brush or comb portion of the applicator will have the hair coloring formulation stored therein while the person conducts a test to determine if the formulation will irritate their skin. Accordingly, if the formulation is not storage stable, the applicator (including the brush/comb portion) must be discarded and a new applicator used to color the hair following the successful testing of the formulation.

U.S. Pat. No. 6,036,005 (Krause et al) discloses a package for storing, mixing and dispensing a multi-component product, such as hair colorings (column 9, line 57). The package of Krause et al includes an outer container for holding a first material and an inner container, and an inner container (interior of the outer container), for holding a second material. When the outer container is squeezed, the inner container is opened and the first and second materials may be mixed together. One disadvantage with the package of Krause et al is that all of the hair coloring formulation would be mixed at once. Accordingly, a user would have to mix all of the formulation, apply a portion of the formulation to their skin to determine if it irritates their skin and subsequently use the remainder of the formulation to color their hair if the formulation does not irritate their skin. However, if the formulation, once mixed, is not stable during this test period, then the remainder of the formulation must be thrown out and a new package opened and used to color the person's hair.

### SUMMARY OF THE INVENTION

The method of the instant invention utilizes an applicator having two storage compartments, wherein each compartment contains a different component of a hair treatment formulation and, preferably, a hair coloring formulation and, most preferably, an oxidative hair dye. The hair coloring formulation may be any formulation, which is a two-component system that requires mixing prior to application to the hair. The hair coloring formulation may be used to color the hair of the user, or merely to tint, streak or highlight hair, and preferably to treat new hair growth to appear essentially the same as existing treated (e.g. coloured) hair.

In a particularly preferred embodiment, the hair coloring formulation is used to color new grown hair at the base of the skull (hair shaft) after hair has been colored. After hair is colored, the new hair that grows is the original color and if not colored, will draw attention to the fact that a person has colored hair. Accordingly, it is desirable to treat the new grown hair. In accordance with this aspect of the invention, the applicator is preferably sized to contain a sufficient amount of the hair coloring formulation to treat such new grown hair once. Accordingly, the volume of the hair coloring formulation in the applicator may be from 5 to 20 ml and preferably 10 to 15 ml. It will be appreciated that if the applicator is intended to have a sufficient amount of the hair coloring formulation to color all of the hair of a person, that the volume of the hair coloring formulation in the applicator may be from 10 to 40 ml and preferably 10 to 20 ml.

The applicator may be any applicator known in the art that stores the two components separately and is preferably plastic. The applicator is provided with a mixer nozzle. In operation, the mixer nozzle is in flow communication with each compartment of the applicator so that the two components enter the mixer nozzle and are combined together as they flow through the mixer nozzle. The outlet of the mixer nozzle therefore delivers a formulation wherein the two components have been mixed together. Such mixer nozzles are known in the art and any such mixer nozzle may be used in accordance with the instant invention. Preferably, the applicator is a dual barrel syringe, such as that of Sylverstein et al, that is provided with a static mixer nozzle. It will be appreciated that the method and kit of the instant invention may be used with a formulation that comprises more than two components. In such an embodiment, the applicator will have more than two compartments. For example, if the formulation is a three-component system, then the applicator will have three components. It will be appreciated that each component may be provided in only one compartment or that a particular component may be provided in two or more compartments provided that the different components are stored in separate compartments. Preferably, the mixer nozzle is polypropylene. An advantage of the use of polypropylene is that it is less reactive with peroxide (which is typically used in a developer for an oxidative hair dye).

The hair coloring formulation is a two component system, which preferably comprises a developer and a dye. More preferably, the hair coloring formulation is an oxidative hair coloring formulation. The dye has a sufficient amount of antioxidant to permit the applicator, once filled, to be stored for an extended period of time, preferably at least 6 months and, more preferably at least 12 months. The amount of antioxidant that is required will vary depending upon several factors, including the amount of oxygen present in the applicator and the packaging of the applicator, the oxygen permeability of the applicator, the color of the dye (darker colors requiring more antioxidant). With the use of a plastic applicator, preferably the dye comprises from 0.6 to 0.2 weight percent antioxidant and, more preferably about 0.4 weight percent antioxidant. Any antioxidant known in the industry may be used.

Alternately, or in addition, the exit of the compartments of the syringe is preferably sealed, such as by being filled with a wax, such as paraffin wax. The wax seals, or assists in sealing, the exit of the compartments once the compartments have been filled. For example, the outlet of the compartments may be dipped in liquid paraffin and then the mixer nozzle attached. When the applicator is used, dispensing an aliquot of hair coloring formulation from the applicator will flush the wax through the mixer nozzle. A consumer preferably does not use this portion of the hair coloring formulation. By sealing the exits of the compartments, the components of the hair coloring formulation will not leak out of the mixer nozzle. The wax forms a rupturable seal that may be broken by the force applied to the plunger of the syringe. Any material that can be deformed in shape to flow through the mixer nozzle, such as a grease, thin plastic strip of the like, may be used in place of the wax.

Preferably, each of the developer and the dye have an apparent viscosity that is sufficiently high to prevent each of the developer and the dye from leaking out of the applicator (e.g., through the seal between the plunger and the fluid chamber in the applicator) and sufficiently low to permit the developer and the dye to mix as they pass through a mixer nozzle. Typically, the dye component is paste like. However, the developer has a low viscosity. In accordance with one aspect of the instant invention, the composition of the developer is adjusted such that, after passing through the mixer nozzle, the hair coloring formulation will be substantially mixed (e.g. 70% or more mixed). As the hair coloring formulation is brushed into the hair of the user after being extruded from the mixer nozzle, the mixing of the hair coloring formulation may be completed. The required viscosity of the developer for the developer and the dye component to be mixed as they pass through the mixer nozzle will depend on several factors including the number of elements (e.g., half helixes) in the mixer nozzle, the diameter of the mixer nozzle containing the elements (e.g., the cross sectional area of the spiral flow passage) and the viscosity of the dye component. Preferably, the developer is thixotropic. This is achieved by adding a thixotrope to the developer. Accordingly, the developer will tend to remain in a storage compartment of the syringe until the plunger is depressed and will have a sufficient apparent viscosity so as to mix with the dye component when the plunger is depressed. Preferably, when flowing, the developer has an apparent viscosity from 4,000 cps to 7,500 cps, and more preferably from 4,500 cps to 5,500 cps.

In accordance to one aspect of this invention, a test portion of the hair coloring formulation (e.g. up to 25%, more preferably up to 15% and, more preferably up to 10%) is mixed, while it is extruded, from the applicator. This portion of the formulation is then applied to any part of the skin of the user as is known in the art. It will be appreciated that the amount that is withdrawn from the barrels of the syringe must be sufficient to fill the mixer nozzle and to provide an aliquot of the formulation sufficient for testing on the skin of the user. Preferably, before testing, an amount equal to the contents of the mixer are extruded and discarded to ensure that the two components are flowing into the mixer simultaneously. If the outlets from the storage compartment are sealed by, e.g., wax, then the first aliquot extruded from the mixer nozzle is preferably discarded. The applicator is preferably stored such that the components of the hair colouring formulation are not exposed to oxygen during the test period. Accordingly, the application may be sealed during the test period such as by placing a closure cap on the mixer nozzle or the barrels of the syringe (if the mixer nozzle is removable), and/or the applicator may be placed in an oxygen impermeable container, e.g., a sealable foil pouch. Subsequently, if the user does not react to the formulation, the applicator may be unsealed and/or removed from the foil pouch and a further portion of the formulation, and preferably all of the remaining formulation, is mixed while extruded from the applicator. It will be appreciated that the user may apply the mixed formulation that is extruded from the applicator directly to their scalp, e.g., if an applicator brush is provided.

The applicator is preferably stored such that the components of the hair colouring formulation are not exposed to oxygen prior to the test portion of the formulation being applied to the skin of the user. For example, the compartments of the applicator may be flushed with nitrogen prior to being filled and the applicator may be shipped in an oxygen reduced environment (e.g. a nitrogen flushed, oxygen impermeable package). Preferably, the applicator is sealed in an air impermeable container, such as a metallic foil pouch. During the manufacture of the product, the air impermeable container is preferably subjected to a nitrogen gas purge to obtain a gas in the packaging that has an oxygen reduced content and, preferably, the gas in the packaging is essentially nitrogen. Accordingly, the individual components of the hair coloring formulation will be able to be stored without any significant degradation while the user tests the formulation on the user's skin.

In a particularly preferred embodiment, a package comprises a single applicator and a single nozzle and, more preferably, the nozzle is mounted to the applicator when purchased by a consumer. Accordingly, on opening a package (e.g. opening a nitrogen purged foil pouch), the consumer may merely remove an optional cap from the end of the mixer nozzle and dispense a small aliquot of formulation to flush wax from the mixer nozzle, if utilized, and then commence use of the formulation to treat their hair and, preferably, just the roots of the hair. Preferably both a cap for the mixer nozzle and a wax filling of at least part of the nozzle it utilized. However, it will be appreciated that only one method may be used to close the applicator during transport in its packaging.

One advantage of such applicators is that the user may dispense only a portion of the formulation that is provided and store the remainder for after the test period. If the user does not react to the formulation, then the remainder of the formulation may be dispensed. A further advantage is that the formulation may be automatically mixed as it is dispensed.

### BRIEF DESCRIPTION OF THE DRAWINGS

These and other advantages of the instant invention will be more fully and clearly understood in connection with the following description of the preferred embodiments of the invention in which:

FIG. 1 is a partially cut away perspective view of a kit according to a first embodiment of the instant invention;

FIG. 2 is a perspective view of the kit of FIG. 1 in use;

FIG. 3 is a partially cut away perspective view of a kit according to an alternate embodiment of the instant invention;

FIG. 4 is a cut away perspective view of a kit according to a further alternate embodiment of the instant invention;

FIG. 5 is a perspective view of a mixing nozzle being attached to the dual barreled syringe shown in FIG. 1;

FIG. 6 is an enlarged view showing the mixer nozzle attached to the dual barreled syringe of FIG. 1;

FIG. 7 is a perspective view of the closure cap when removed from the end of the dual barreled syringe of FIG. 1;

FIG. 8 is an end view of the closure cap of FIG. 7;

FIG. 9 is an end view of the dual barreled syringe of FIG. 7; and,

FIG. 10 is a cross section through the closure cap and the dual barreled syringe along the line 10-10 shown in FIG. 1.

### DETAILED DESCRIPTION OF THE INVENTION

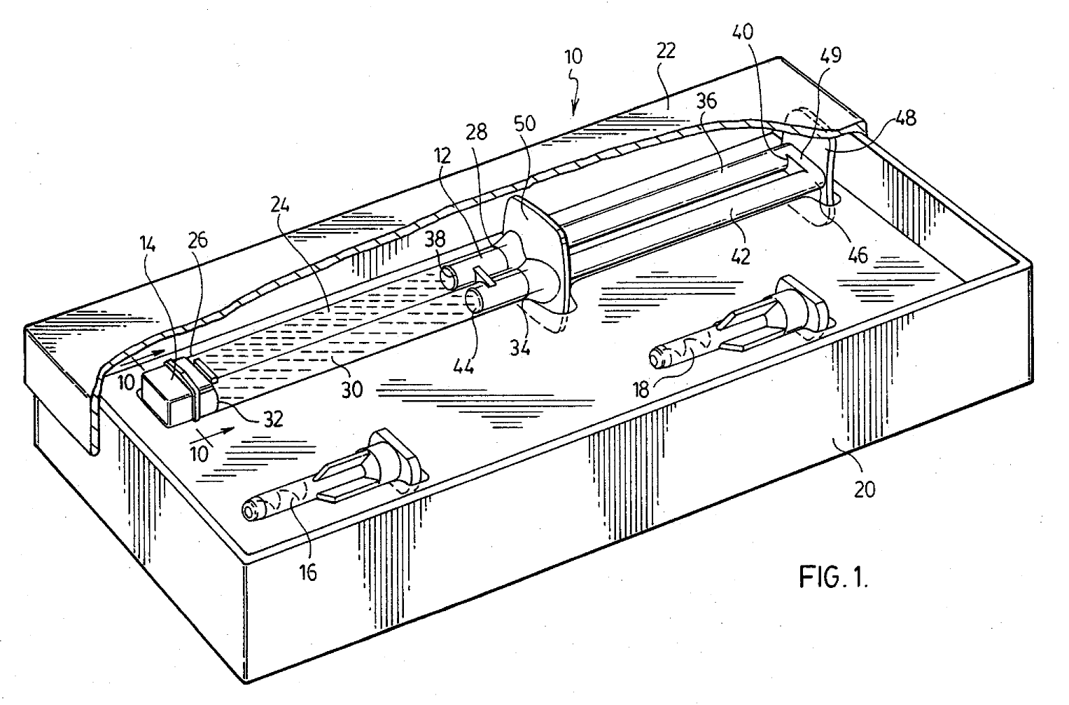

As shown in FIG. 1, a kit 10 according to one preferred embodiment of the instant invention comprises an applicator 12, a closure member 14 and first and second dispenser nozzles 16 and 18.

The kit may be provided in any packaging and, preferably, a packaging suitable for retail sale. For example, as shown in FIG. 1, the kit 10 is provided in a package having a bottom 20 that is provided with recesses for removably receiving therein the components of the kit, and a top 22, a portion of which is broken away in FIG. 1 so as to reveal the contents of the container. It will be appreciated that all or a portion of top 22 may be transparent (e.g. it could be made from thin plastic). It will also be appreciated that the packaging could be a blister pack or other package known in the art.

Applicator 12 may be any applicator known in the art, which has at least as many compartments as the formulation to be mixed. Preferably, the formulation is a two-component system and applicator 12 has two storage compartments—one for each component. For example, applicator 12 may have two storage compartments wherein the contents of each of the storage compartments is emptied at the same time when a user actuates a dispenser or squeezes on the storage compartments.

In one embodiment, the storage compartments may be individually formed and then assembled into a frame so as to form a single applicator 12. In another embodiment, the dispenser nozzle itself may provide the frame or holder for receiving individually formed storage compartments. Most preferably, applicator 12 comprises a dual barreled syringe.

As shown in FIG. 1, a dual barreled syringe has a first storage compartment or barrel 24 having a first end 26 and a second end 28 and a second compartment or barrel 30 having a first end 32 and a second end 34. Applicator 12 also includes a first plunger 36 having a first end 38 and a second end 40, and a second plunger 42 having a first end 44 and a second end 46. Plungers 36, 42 may be connected together so that each plunger is actuated at the same time. Accordingly, a cross member 49 and/or a first flange 48 may connect the second ends 40, 46 of plungers 36, 42 together. In order to secure the barrels 24, 30 together, a second flange 50 may be provided. Second flange 50 may be secured to send ends 28, 34 of barrels 24, 30. Accordingly, when assembled together, barrels 24, 30, plungers 36, 42 and flanges 48, 50 may create a one-piece assembly. It will be appreciated that barrels 24, 30 may be co-extruded so as to be integrally formed. Similarly, plungers 36, 42 may be integrally formed with cross member 49, and optionally together with flanges 48 and/or 50. Any other construction technique known in the art may be used.

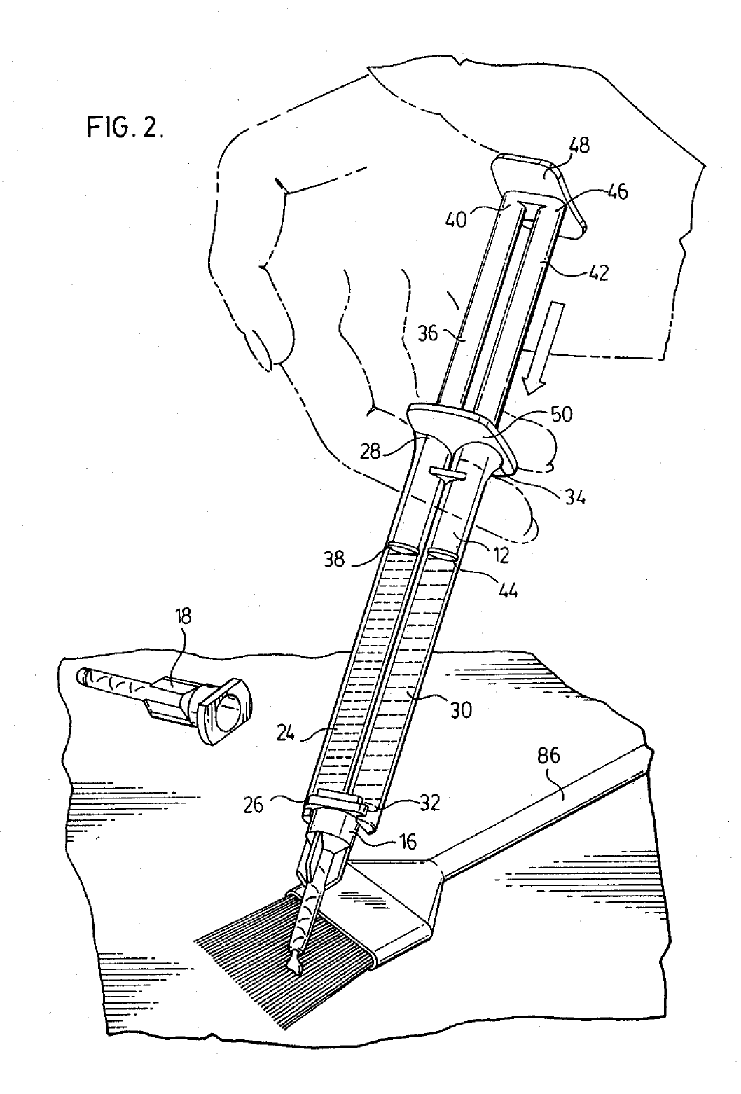

In operation, a user may grasp flanges 48 and 50 as shown in FIG. 2 and apply pressure to flange 48 so as to force plungers 36, 42 to pass through barrels 24, 30 thereby dispensing the components in barrels 24, 30.

If the formulation is an oxidative hair coloring formulation, then the dye component may be provided in barrel 24 and the developer may be provided in barrel 30. The dye component and developer may be designed to be mixed in a one to one ratio. As such, barrels 24, 30 may have the same volume and may be dispensed at the same rate. It will also be appreciated that if the dye component and developer are to be mixed in a different ratio, that applicator 12 could be configured to dispense the developer and the dye component at different rates so that the dye component and developer are dispensed in the required ratio. It will also be appreciated that the volume of the barrels may differ, such as by varying the length and/or diameter of barrels 24, 30.

Preferably, each of the developer and the dye have an apparent viscosity that is sufficiently high to prevent each of the developer and the dye from leaking out of the applicator and sufficiently low to permit the developer and the dye to mix, or at least substantially mix, as they pass through a mixer nozzle. For example, the first ends 38, 44 of plungers 36, 42 may be provided with an O-ring or a flange may be formed as part of plungers 36, 42 to permit the first ends 38, 44 of plungers 36, 42 to contact the inner wall of the fluid chambers 24, 30. While the first ends 38, 44 of plungers 36, 42 may contact the inner wall of the fluid chambers 24, 30, if the viscosity the dye or of the developer is too low, fluid may leak therethrough. Further, if the viscosity is too high, then the dye and the developer will not mix as the dye and developer are extruded through nozzle 16. Preferably each of the developer and the dye has an apparent viscosity from 4,000 cps to 7,500 cps, and more preferably from 4,500 cps to 5,500 cps. In addition, it is preferred that the developer is thixotropic.

Preferably, the hair coloring formulation is an oxidative hair coloring formulation. The dye has a sufficient amount of antioxidant to permit the applicator, once filled, to be stored for an extended period of time, preferably at least 6 months and, more preferably at least 12 months. The amount of antioxidant that is required will vary depending upon several factors, including the amount of oxygen present in the applicator and the packaging of the applicator, the oxygen permeability of the applicator, the color of the dye (darker colors requiring more antioxidant). With the use of a plastic applicator, preferably the dye comprises from 0.6 to 0.2 weight percent antioxidant and, more preferably about 0.4 weight percent antioxidant. Any antioxidant known in the industry may be used.

Dispenser nozzles 16, 18 are adapted to be attached to dual barreled syringe 12. It will be appreciated that in accordance with one embodiment of this invention, dispenser nozzle 16 may be removably attachable to dual barrel syringe 12 and dispenser nozzle 18 may be non-removably attachable to dual barreled syringe 12 (i.e. once attached to dual barreled syringe 12, it is not designed to be removed therefrom). Preferably, each dispenser nozzle 16, 18 is removably attachable to dual barreled syringe 12.

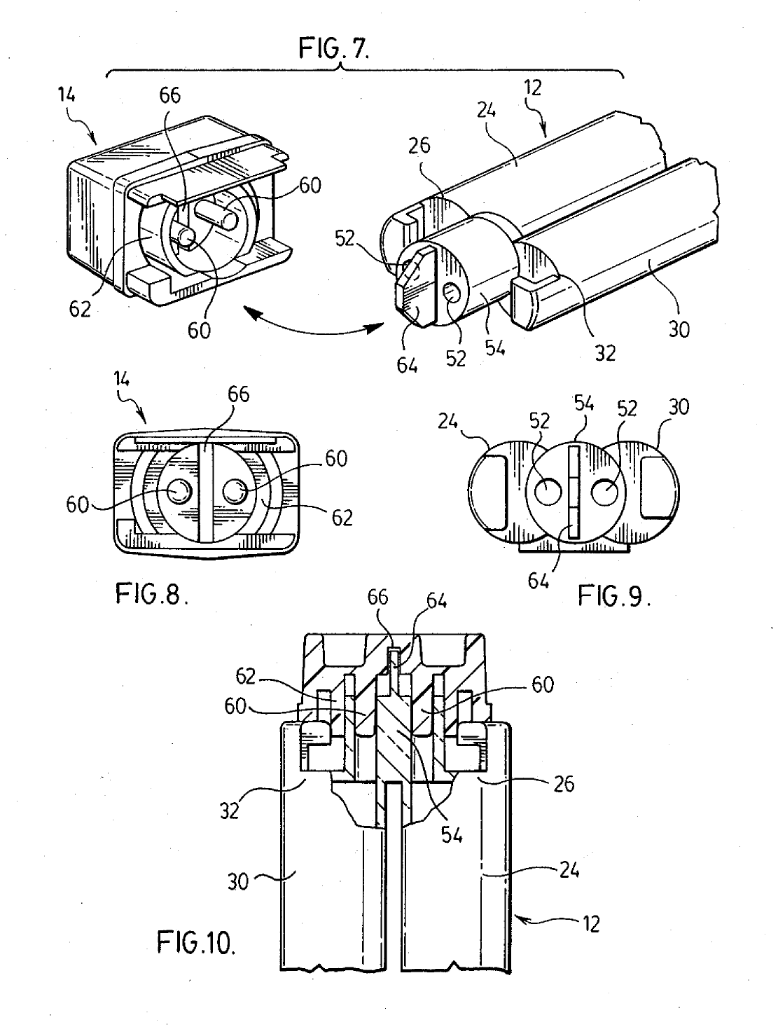

Each of barrels 24, 30 has an outlet 52. For example, as shown in FIGS. 7 and 9, first ends 26, 32 of barrels 24, 30 are each provided with at least one outlet 52. As shown therein, outlets 52 extend through housing 54 so as to be in fluid flow communication with the interior of barrels 24, 30. It will be appreciated that outlets 52 may be of any particular configuration and may be provided directly on ends 26, 32 of barrels 24, 30.

When dual barreled syringe 12 is acquired by a user, dual barreled syringe 12 is preferably sealed and, more preferably, outlets 52 are sealed. It will be appreciated that any member which seals applicator 12 and, preferably, outlets 52 to prevent the dye component and developer from mixing while still in applicator 12 and to prevent degradation of the dye component and the developer by exposure to the atmosphere may be utilized. For example, as shown in FIG. 1, dual barreled syringe 12 may be provided with a closure member 14 that is removably attachable to dual barreled syringe 12. Alternately, as shown in FIG. 4, dual barreled syringe 12 may be provided in kit 10 with a foil closure member 16 having a pull-tab 58. A user may grasp pull-tab 58 to remove foil closure cap 56 to open dual barreled applicator 12. Any such closure member known in the art may be used. For example, the closure member need not be made from foil but may be made from plastic or a water impermeable cellulose substrate. It will be appreciated that this form of closure member is not re-usable. If a disposable closure member is used (e.g., closure member 56), then a closure member 14 that is removably attachable to dual barreled syringe 12 is preferably provided if kit 10 includes a single dual barreled syringe 12. However, if kit 10 comprises two applicators 12, each of which is sealed by a disposable closure member is used (e.g., closure member 56), then kit 10 need not comprise a closure member 14 that is removably attachable to dual barreled syringe 12. It will be appreciated that in accordance with another embodiment of this invention, only a single syringe 12 is provided and a dispenser nozzle is mounted thereon when purchased by a consumer. In such an embodiment, then, if a closure member is provided, the closure member preferably comprises a rupturable seal that may be broken by the force applied to the plunger of the syringe and wherein the member comprising the seal may pass through the mixer nozzle as the plunger is depressed. Any material that can be deformed in shape to flow through the mixer nozzle, such as a grease, thin plastic strip of the like, may be used in place of the wax.

It will be appreciated that the interior of dual barreled syringe 12 is preferably purged with nitrogen prior to the dual barreled syringe being filled and, in addition, the container for the syringe. In such a case, the dual barreled syringe may optionally also be sealed as well as being stored in a nitrogen environment.

FIGS. 7 and 8 show a preferred embodiment of a resealable cap 14. As shown therein, the rear face of closure cap 14 has two sealing members 60. Each sealing member 60 is adapted to be received in an outlet 52 thereby sealing outlets 52. In order to secure closure member 14 to dual barreled syringe 12, the rear face of closure member 14 may be provided within annular member 62 that surrounds outlets 60. Annular member 62 is preferably sized so as to be slidingly received on the outer surface of housing 54. Frictional engagement between the inner wall of annular member 62 and the outer wall of housing 54 may be used to maintain closure member 14 secured to housing 54. Alternately, or in addition, other securing means know in the art may be used such as male and female engagement members, one of which is provided on closure member 14 and the other of which is provided on syringe 12. Optionally an alignment member 64 may be provided on housing 54 and an opening or slot 66 may be provided in closure members 14 for removably receiving alignment member 64. Optional alignment member 64 aids in guiding sealing members 60 into outlets 52 as closure member 14 is provided on housing 54.

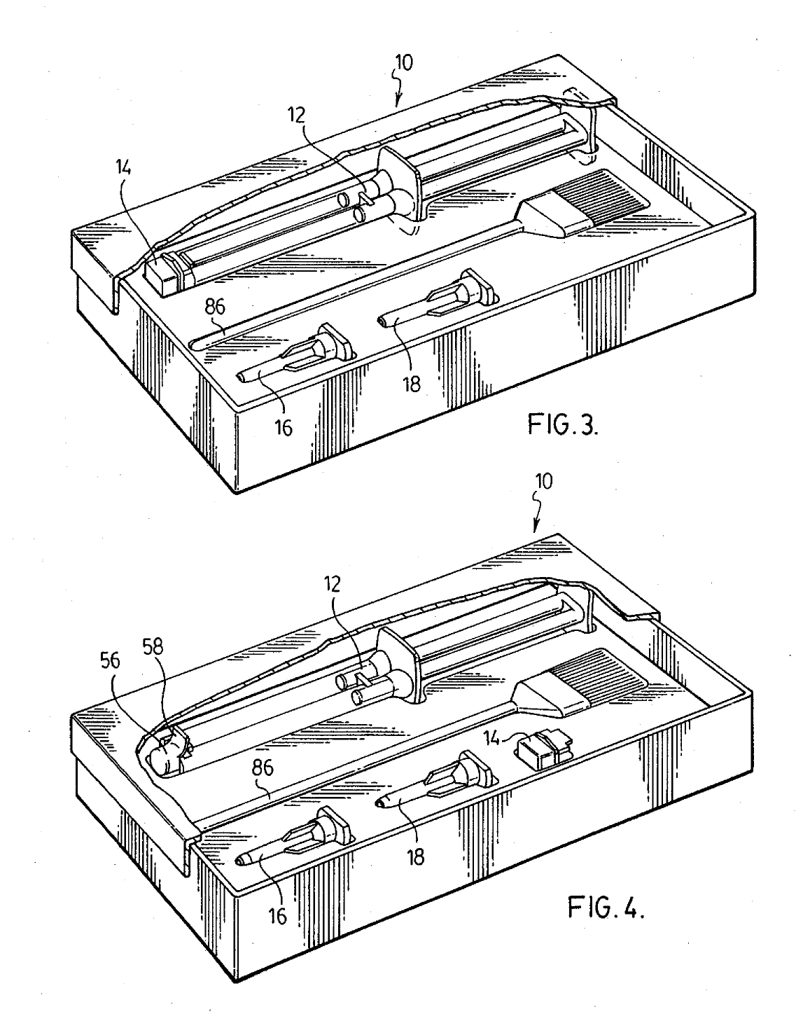

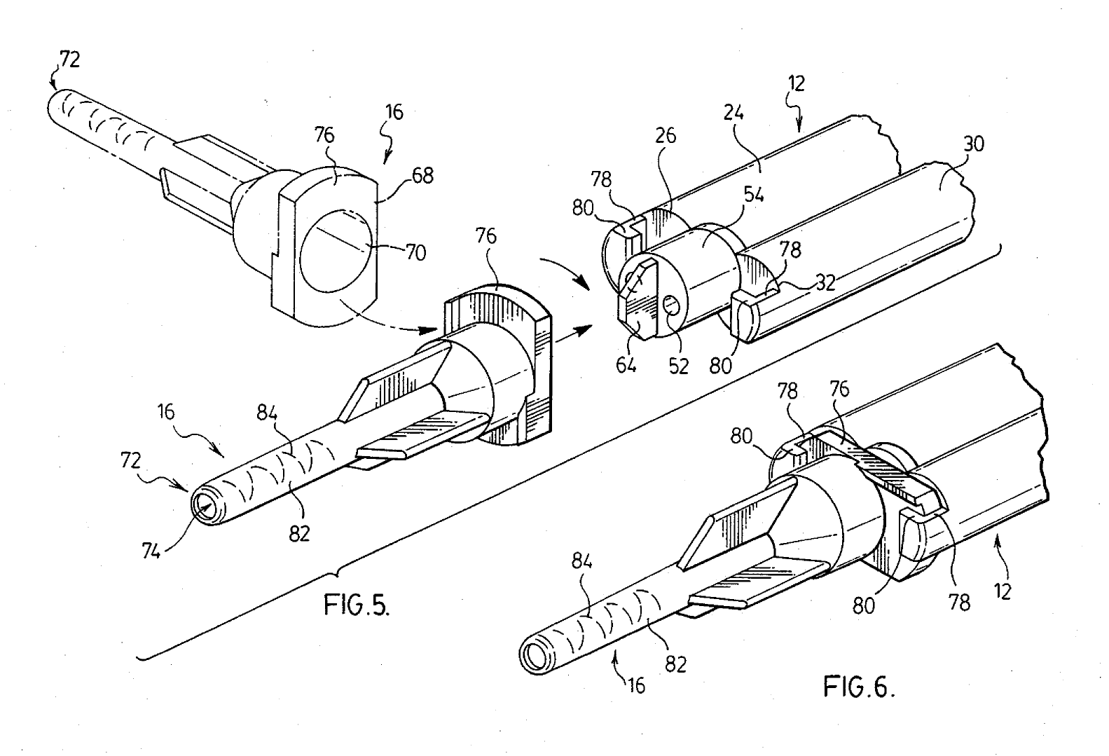

Dispenser nozzles 16, 18 preferably are configured to mix the dye component and developer together as they flow through dispenser nozzle 16, 18. Accordingly, dispenser nozzles 16, 18 are preferably static mixer nozzles. As shown in FIGS. 5 and 6, nozzles 16, 18 preferably have an inlet end 68 having an opening 70 sized to receive at least a portion of housing 54, an outlet end 72 having at least one outlet 74 and a mixer portion 82, which is configured to at least partially mix the components together. For example, mixer nozzle 82 may have an extended flow passage that may be in the form of a helical passage 84. A moveable mechanical member may be provided to assist in mixing the components together as they pass through nozzle 16, 28. More preferably, the formulation is at least essentially mixed when it is dispensed from nozzle 16, 18. By “at least essentially mixed”, it is meant that the formulation is mixed sufficiently so that it may be used without further mixing.

It will be appreciated that outlet end 72 may have a hollow comb or brush applicator receivably mountable on the end thereof or integrally formed thereon. Any applicator comb or brush, which is designed to have a fluid flow therethrough, may be provided on or with nozzle 16, 18. Preferably, two such applicator combs or brushes are provided.

Nozzle 16, 18 may be receivably mounted on dual barreled syringe 12 by any means known in the art. For example, as shown in FIG. 5, inlet end 68 of nozzle 16, 18 is provided with a flange 76. Dual barreled syringe 12 is provided with flanges 78, the distal end of each of which has an arm member 80 provided thereon. Accordingly, nozzle 16, 18 may be aligned with housing 54 as shown in FIG. 5 and then moved towards housing 54 so as to receive housing 54 in opening 70. Subsequently, nozzle 16, 18 may be rotated 90° relative to dual barreled syringe 12 to the position shown in FIG. 6 such that arms 80 engage flange 76. Accordingly, nozzle 16, 18 is removably secured to dual barreled syringe 12. It will be appreciated that nozzle 16, 18 may be engagably secured and, preferably, releasably secured to dual barrel syringe 12 by any other means known in the art.

It will also be appreciated that, in accordance with another embodiment of the instant invention, the applicator may have a nozzle 16 provided thereon when the applicator is purchased by a user. Nozzle 16 may be permanently attached to the applicator or removable mounted thereon. In such a case, the syringe with applicator attached may be sold in a package that contains only the pre-filled applicator with nozzle 16 attached. In accordance with such an embodiment, a user need not have to assemble the application but may merely use a preassembled applicator.

Preferable, the applicator or kit is provided in a package, which was subjected to a purge to remove or reduce the oxygen in the package, prior to the package being sealed.

In use, the components in barrels 24, 30 are mixed as they flow through nozzles 16, 18. In the embodiment shown in FIGS. 5 and 6, when the components are dispensed from barrels 24, 30 via outlets 52, the components enter mixer portion 82. Accordingly, when the components exit outlet 74, the dye component and developer are preferably essentially mixed (i.e. preferably no additional mixing is required prior to use of the hair coloring formulation).

The use of kit 10 will now be discussed. In the embodiment of FIG. 1, kit 10 includes a single applicator 12. When a user acquires kit 10, outlets 52 of barrels 24, 30 are preferably sealed, such as by removable closure member 14. Further, kit 10 may be in an oxygen impermeable container. The user will open the oxygen impermeable container, if provided, and/or remove closure member 14 (e.g. by pulling closure member 14 off of housing 52) as may be required. First dispenser nozzle 16 may then be secured to applicator 12 such as by the mechanism shown in FIGS. 5 and 6. The user may then force plungers 36, 42 partially into barrels 24, 30 so as to mix while dispensing a first aliquot or amount (e.g., 20 volume percent or less) of the hair coloring formulation. The formulation may be dispensed onto a brush or other surface, or directly on to some of the hair or skin of the user. Preferably, a small amount of the formulation is dispensed and allowed to remain in contact with the skin of the user for a sufficient amount of time to determine if the scalp of the user will react to the hair coloring formulation. After the first aliquot of formulation has been dispensed, nozzle 16 is preferably removed and discarded. Closure member 14 is then re-applied to dual barreled syringe 12 to seal barrels 24, 30. Subsequently, e.g., after a sufficient amount of time has passed for a user to determine if the skin of the user reacts to the hair coloring formulation, the user may again remove closure member 14 and install second dispenser nozzle 18. The user may then dispense an additional amount of the hair coloring formulation and use the hair coloring formulation as needed.

In accordance with an alternate method of the instant embodiment, it will be appreciated that instead of providing two dispenser nozzles 16, 18 that a kit 10 may contain a single removably mountable dispenser nozzle 16. In accordance with this embodiment of the invention, after the first aliquot of hair coloring formulation has been dispensed, the user cleans applicator 16 to remove the hair coloring formulation therein. Accordingly, if the skin of the user does not react to the hair coloring formulation, the user may remove closure member 14 and re-install dispenser nozzle 16. It will also be appreciated that this method may be used with the kit of FIG. 1.

In accordance with the alternate embodiment shown in FIG. 3 of the instant invention, a kit 10 may comprise an applicator 12, two closure members 14 and two nozzles 16, 18. Brush 86 may optionally be provided. In accordance with this embodiment of the invention, after the oxygen impermeable container, if provided, is opened, a user may remove closure member 14 from applicator 12 and discard closure member 14. First dispenser nozzle 16 may then be attached to applicator 12 and a first aliquot of the hair coloring formulation dispensed. Nozzle 16 may then be discarded and second closure member 14 affixed to applicator 12. Subsequently, the second closure member 14 may be removed and second dispenser nozzle 18 installed and additional amounts of the hair coloring formulation may be dispensed. The provision of a second closure member may be advantageous to prevent or reduce the likelihood of some of the dye component being contaminated with developer while in applicator 12. For example, if some of the developer is on a sealing member 60 or is within the opening defined by annular member 62, then some of the developer may be inserted into the outlet 52 of the dye component when closure cap 14 is re-applied to applicator 12. The use of a clean closure member 14 will prevent such contamination.

In accordance with the alternate embodiment shown in FIG. 4, kit 10 comprises an applicator 12, a foil closure member 56, two nozzles 16, 18 and a removably attachable closure member 14. An optional brush 86 may be provided. In accordance with this embodiment, the method comprises opening the oxygen impermeable container, if provided, removing and disposing of closure member 56 and attaching dispenser nozzle 16 to applicator 12. After a first aliquot of the hair coloring formulation has been dispensed, nozzle 16 is removed and discarded and closure member 14 is applied to applicator 12. Subsequently, closure member 14 may be removed and second dispenser nozzle 18 installed on applicator 12. Further amounts of the hair coloring formulation may then be dispensed. In a variation of this method, it will be appreciated that kit 10 of FIG. 4 may comprise a single nozzle 16 and, accordingly, a user may wash out applicator 16 prior to the re-use of nozzle 16.

In accordance with a further alternate embodiment of the instant invention, it will be appreciated that a kit 10 may comprise two applicators 12, each of which is sealed, and at least one dispenser nozzle 16 and, preferably, two dispenser nozzles 16, 18. One or both of the applicators 12 may be sealed with a removable closure member 14 or a foil closure member 56. In accordance with such an embodiment, a user may open the oxygen impermeable container, if provided and then take a first applicator 12, remove the closure member and install a nozzle 16. The user may then dispense an aliquot of the hair coloring formulation for testing purposes. The applicator 12 may then be disposed of with or without nozzle 16. If the applicator 12 is disposed of with nozzle 16, then kit 10 is provided with a second nozzle 18. After the test period, the user may then take the second applicator 12, remove the closure member and install the second nozzle 16 (or a washed first nozzle 16). Additional amounts of hair coloring formulation may then be dispensed for use.

In accordance with this alternate embodiment, it will be appreciated that since each applicator 12 is disposed of after use, kit 10 may include two nozzles 16, 18 that need not be removably mountable on applicators 12. For example, nozzles 16, 18 may be non-removably engagable with applicator 12, provided already installed on one or each applicator 12 or integrally formed as part of each applicator 12. If nozzles are provided already installed or integrally formed as part of applicators 12, then a means is provide to permit outlets 52 to be opened. For example, each nozzle 16, 18 may have a valve formed therein. Alternately, a foil closure member 56 may be provided to cover outlets 52 and a pull-tab 58 may extend out beyond nozzle 16. Any other opening means known in the art may be used.

In accordance with this alternate embodiment, applicators 12 may contain different amounts of the hair coloring formulation. In particular, one of the applicators 12 may contain only a sufficient amount of the hair coloring formulation to permit the user to test the formulation on the skin of the user. The other applicator 12 may contain the required amount to permit the user to color their hair. Accordingly, one of the applicators may contain about 20%, and, more preferably, about 10% of the amount of the hair coloring formulation in the other applicator.

In accordance with a further alternate embodiment of the instant invention, it will be appreciated that a kit 10 may comprise a single applicator 12 which has a nozzle 16 attached thereto. Kit 10 may sealed, such as by providing application 12 and nozzle 16 in an air impermeable container (e.g., a foil pouch). Alternately, or in addition, and preferably in addition, nozzle 16 is sealed, such as by a closure member on the end of nozzle 16 distal to barrels 24, 30. In use, the user may open an optional nitrogen purged pouch, dispense an aliquot of the hair coloring formulation to remove the optional rupturable seal and an additional aliquot for testing purposes. The applicator 12 and nozzle 16 may then be stored, such as by being sealed in the air impermeable container, which is preferably resealable by any means known in the art, and/or by placing a cap on the end of nozzle 16. After the test period, the user may then take the applicator 12, remove the cap and/or open the air impermeable container and dispense additional amounts of hair coloring formulation.

It will be appreciated by those skilled in the art that various additions and modifications may be made to the kit and the method disclosed herein and all such modifications and additions are within the scope of the following claims. In particular, the method of attachment of nozzle 16, 18 to applicator 12, the method of attachment of closure members 14, 56, the construction of the nozzles, the applicator, the closure members or the brush may be any of those known in the art. It will also be appreciated that the hair treatment formulation may be any two-component dye, tint or other hair treatment known in the art. In addition, the packaging for kit 10 may be of any design.

1. An apparatus for coloring at least a portion of the hair of a person comprising using a formulation prepared by mixing together at least first and second components, the apparatus comprising:
a) an applicator comprising
i) at least one compartment for each component, each compartment having an outlet; and,
ii) a dispenser nozzle in fluid communication with the compartments, the dispenser nozzle having an exit port,
b) each component having an apparent viscosity selected to prevent each component from leaking out of its compartment and to permit the components to be at least substantially mixed together as the components pass through the dispenser nozzle.

2. The apparatus as claimed in claim 1 wherein the first component comprises a developer and the second component comprises a dye.

3. The apparatus as claimed in claim 2 wherein the formulation is an oxidative hair dye.

4. The apparatus as claimed in claim 1 wherein each component has an apparent viscosity above about 4,000 cps.

5. The apparatus as claimed in claim 2 wherein each component has an apparent viscosity from about 4,000 cps to about 7,500 cps.

6. The apparatus as claimed in claim 1 wherein the applicator is plastic and the second component comprises a dye and the second component comprises a sufficient amount of antioxidant to prevent oxidation of the second component for at least one year.

7. The apparatus as claimed in claim 6 wherein the second component comprises from 0.6 to 0.2 weight percent antioxidant.

8. The apparatus as claimed in claim 6 wherein the second component comprises about 0.4 weight percent antioxidant.

9. The apparatus as claimed in claim 6 wherein the second component comprises a red, brown or black dye.

10. The apparatus as claimed in claim 6 wherein the second component comprises a blond dye.

11. The apparatus as claimed in claim 1 further comprising a rupturable seal provided at the outlets of the compartments.

12. The apparatus as claimed in claim 11 wherein the rupturable seal comprises a wax or a grease.

13. The apparatus as claimed in claim 1 further comprising a closure cap releaseably mounted to the exit port of the nozzle.

14. The apparatus as claimed in claim 1 wherein the applicator is provided in a nitrogen purged package.

15. The apparatus as claimed in claim 14 wherein the package is air impermeable.

16. The apparatus as claimed in claim 1 wherein the applicator contains an amount of the formulation that is sized to treat only the new growth of a person hair.

17. The apparatus as claimed in claim 16 wherein the applicator contains from 5 to 20 ml of the formulation.

18. The apparatus as claimed in claim 1 wherein the mixer nozzle is made from polypropylene.

19. An apparatus for coloring at least a portion of the hair of a person comprising using a formulation prepared by mixing together at least first and second components, the apparatus comprising:
a) an applicator comprising
i) at least at least one compartment for each component, each compartment having an outlet; and,
ii) a dispenser nozzle in fluid communication with the compartments, the dispenser nozzle having an exit port,
b) a rupturable seal provided at the outlets of the compartments.

20. The apparatus as claimed in claim 19 wherein the rupturable seal comprises wax.

21. An apparatus for coloring at least a portion of the hair of a person comprising using a formulation prepared by mixing together at least first and second components, the apparatus comprising:
a) an applicator comprising
i) at least at least one compartment for each component, each compartment having an outlet; and,
ii) a dispenser nozzle in fluid communication with the compartments, the dispenser nozzle having an exit port,
wherein the applicator is provided in a nitrogen purged package.

22. The apparatus as claimed in claim 21 wherein the compartments are purged with nitrogen prior to being filled with the first and second components.
